SEQUENCE LISTING (6 sequences)

SEQ ID NO: 1 - DNA, 33 bp, Homo sapiens
atcggatcctattatgtgtccaccagccctaag

SEQ ID NO: 2 - DNA, 32 bp, Homo sapiens
acctctagacatcagcccagactggaatgctg

SEQ ID NO: 3 - DNA, 60 bp, Homo sapiens
ggaaaactcgagatggattacaaggatgatgatgataagaaggccaaggaacccttgccc

SEQ ID NO: 4 - DNA, 33 bp, Homo sapiens
ggaaaatctagaacgaaaacgactagactccag

SEQ ID NO: 5 - DNA, 34 bp, Homo sapiens
atcaggatccctacagacccttcctcctgtccca

SEQ ID NO: 6 - DNA, 34 bp, Homo sapiens
gtacgaattcatcagcccagactggaatgctgca

(19) United States
(12) Patent Application Publication
(10) Pub. No.: US 2003/0109501 A1
(43) Pub. Date: Jun. 12, 2003

(54) High throughput assay for N-sulfotransferase activity of glucosaminyl N-deacetylase/N-sulfotransferases

(76) Inventors: Guchen Yang, Morrisville, PA (US); Peter A. Kiener, Doylestown, PA (US); Henry Shen, Princeton, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/263,970

(22) Filed: Oct. 3, 2002

Related U.S. Application Data
(60) Provisional application No. 60/328,080, filed on Oct. 5, 2001.
(60) Provisional application No. 60/331,832, filed on Nov. 20, 2001.

(51) Int. Cl. A61K 31/66; C12Q 1/34

(52) U.S. Cl. 514/102; 435/18

(57) ABSTRACT

Assays for N-sulfotransferase activity of glucosaminyl N-deacetylase/N-sulfotransferases (NDSTs) include transferring a radiolabeled sulfate from a sulfate donor to a polysaccharide acceptor in the presence of an NDST enzyme to form a radiolabeled sulfated polysaccharide, the radiolabeled sulfated polysaccharide which when bound to a potentially scintillating particulate within an activating distance emits a detectable signal indicative of N-sulfotransferase activity. The assay provides high throughput assays for screening inhibitors that block N-sulfotransferase activity of an NDST. Specific compounds that inhibit N-sulfotransferase activity of an NDST and therapeutic uses of these compounds are disclosed.

This application claims benefit to provisional application U.S. Serial No. 60/328,080, filed Oct. 5, 2001; and to provisional application U.S. Serial No. 60/331,832, filed Nov. 20, 2001. The entire teachings of the referenced applications are incorporated herein by reference.

### FIELD OF THE INVENTION

The present invention relates to a scintillation proximity assay (SPA) for high throughput screening of compounds capable of inhibiting N-sulfotransferase activity of glucosaminyl N-deacetylase/N-sulfotransferases (NDSTs).

### BACKGROUND OF THE INVENTION

Mast cells are one of the major effector cells in the pathogenesis of asthma and other allergic diseases. They contain numerous cytoplasmic granules that are packed with fully activated inflammatory mediators, such as histamine and mast cell proteases. Heparin is synthesized exclusively by mast cells and stored in the secretory granules in complex with these fully activated inflammatory mediators. These mediators are known to cause allergic reactions leading to the incidence of asthma and other allergic diseases. In asthma, once mast cells are triggered by allergens, the granules burst causing bronchoconstriction, plasma exudation, and allergic inflammation [Busse, W. W. and Lemanske, R. F. (2001) N. Engl. J. Med. 344,350-62]. Glucosaminyl N-deacetylase/N-sulfotransferase-2 (NDST-2) is the rate-limiting enzyme of heparin modification in mast cells and is required for granule formation [Karlinsky, J. B. et al. (1998) Biochem. J. 332,303-307]. Mast cells from NDST-2-deficient mice lack normal granules; these cells lack the biosynthesis of sulfated heparin and contain significantly less histamine and proteases [Humphries, D. E. et al. (1999) Nature. 400,769-772 and Forsberg, E. et al (1999) Nature. 400,773-776]. Inhibitors of NDST-2 are likely to be useful for the treatment of asthma and other allergic diseases.

Moreover, mast cells also release a number of immunoregulatory cytokines such as interleukin-1 (IL-1), IL-3, IL-4, IL-5, IL-6, IL-8, IL-13, tumor-necrosis factor-alpha (TNF-alpha), interferon-gamma and granulocyte-macrophage colony-stimulating factor (GM-CSF) and lipid-derived inflammatory mediators, such as leukotrienes and prostaglandins [Metcalfe, D. D. et al. (1997) Physiol. Rev. 77,1033-1079]. These molecules, as well as histamine and mast cell proteases, have been implicated in the pathogenesis of a variety of clinical conditions associated with local inflammation and tissue remodeling. Therefore, NDST-2 inhibitors are also likely to be useful in the treatment of mast cell-associated inflammatory diseases, including rheumatoid arthritis, chronic obstructive pulmonary disease (COPD), fibrotic lung disease, neurofibromatosis, psoriasis, scleroderma, interstitial cystitis, ulcerative colitis, and Crohn's disease.

Currently, four glucosaminyl N-deacetylase/N-sulfotransferases have been identified; NDST-1, NDST-2, NDST-3 and NDST-4 [Aikawa, J. et al. (2001) J. Biol. Chem. 276,5876-5882]. As described above, NDST-2 is the rate-limiting enzyme in heparin sulfation in mast cells. The biosynthesis of sulfated heparan sulfate requires enzymes NDST-1, NDST-3 or NDST-4. The sulfated heparan sulfate proteoglycans are abundant cell-surface molecules that function as co-receptors in the formation of receptor-signaling complexes. [Bernfield, M. et al. (1999) Annu. Rev. Biochem. 68,729-777]. It has been demonstrated that cell surface heparan sulfate proteoglycans can immobilize basic fibroblast growth factors (bFGF), increase its local concentration, change its conformation, and present it to its signaling receptors [Yayon A. et al. (1991) Cell 64,841-848]. Reduction of the sulfation of the heparan sulfate reduces binding of bFGF to its cell surface receptor, suggesting that the sulfated heparan sulfate is required for bFGF function [Rapraeger, A. C. et al. (1991) Science 252,1705-1708]. The co-receptor role is also applied for the binding of microbial pathogens to host cells [Feyzi, E. et al. (1997) J. Biol. Chem. 272,24850-24857] and the binding of cytokines and chemokines to receptors [Hoogewerf, A. J. (1997) Biochemistry 36,13570-13578]. In addition, heparan sulfate proteoglycans play important roles in cellular adhesion, migration, proliferation, differentiation and angiogenesis. Inhibitors of NDST-1, NDST-3 or NDST-4 are likely to be useful for the treatment of microbial infections, wound repair, inflammatory diseases, and certain neoplastic conditions.

Both N-deacetylase and N-sulfotransferase enzymatic activities are shared with all NDSTs and known enzymatic assays for both N-deacetylase and N-sulfotransferase activities are low throughput [Wei, Z. and Swindler S. J., (1999) J. Biol. Chem. 274, 1966-1970]. N-deacetylase activity is typically measured by determining the release of radioactive ³[H] acetic acid from the ³[H]-labeled Escherichia coli (E. coli.) K5 polysaccharide in the presence of an NDST enzyme. The released ³[H] acetic acid is extracted with ethyl acetate, and the radioactivity is determined. N-sulfotransferase activity is currently measured by incorporation of ³⁵[S]-adenosine 3′-phosphate 5′-phosphosulfate (PAPS) into the deacetylated K5 polysaccharide. The unincorporated PAPS is separated from the ³⁵[S]-sulfated K5 polysaccharide by chromatography. The radioactivity incorporated as sulfated polysaccharide is determined by using a scintillation counter. There are several disadvantages of these procedures, particularly when applied to high-throughput screening for inhibitors as new leads for drug development. First, the radiolabeling of the ³[H]-acetyl polysaccharide requires the complicated preparation of materials. Second, both protocols require separation of radiolabeled polysaccharide from radioactive acetic acid or PAPS by extraction or chromatography. Finally, due to the number of manipulations and steps involved, the protocols are very tedious to conduct manually and are not readily adaptable to robotic automation to increase throughput.

U.S. Pat. No. 4,568,649 (Bertoglio-Matte) discloses an assay for detecting a reactant in a test sample in which beads that are impregnated with a fluorescer are coated with a ligand that binds specifically to a radiolabeled reactant being investigated. In this assay, the portion of the radiolabeled reactant that binds to the ligand is brought in close enough proximity to the beads to activate the fluorescer to produce light energy. The level of light energy produced by the fluorescer is indicative of the amount of reactant present in the test sample. A disadvantage of this assay is that it requires radioactive labeling of the reactant that is being investigated. Many reactants may not be available in radiolabeled form from a commercial source. Moreover, radiolabeling of the reactant may affect its specificity for the ligand coated on the beads.

Therefore, there is a need to provide a method suitable for high throughput screening of compounds that affect NDST activities. In particular, a scintillation proximity assay (SPA) that does not require radiolabeling of the reactant being investigated would be desirable.

### SUMMARY OF THE INVENTION

The present invention provides assays for N-sulfotransferase activity, and/or N-deacetylase activity of an NDST enzyme which includes the step of transferring a radiolabeled sulfate from a sulfate donor to a polysaccharide acceptor in the presence of an NDST enzyme to form a radiolabeled sulfated polysaccharide, the radiolabeled sulfated polysaccharide which when bound to a potentially scintillating particulate within an activating distance emits a detectable signal indicative of N-sulfotransferase activity, and/or N-deacetylase activity.

In one assay, the transferring step includes transferring the radiolabeled sulfate from a sulfate donor to a free polysaccharide sulfate acceptor to form a radiolabeled sulfate polysaccharide and binding the radiolabeled sulfate polysaccharide to the potentially scintillating particulate. The invention further provides an assay where the transferring step includes transferring the radiolabeled sulfate from a sulfate donor to a sulfate acceptor bound to the potentially scintillating particulate to form the radiolabeled sulfate polysaccharide.

In particular, the present invention provides both a solid phase scintillation proximity assay (SPA) and a solution phase SPA assay. The SPA assays are single-step, homogeneous and are ideal for high throughput screening for compounds capable of inhibiting N-sulfotransferase activity, and/or N-deacetylase activity of NDSTs.

In the solid phase SPA assay of the invention, lectin-coated SPA beads, (e.g. WGA-coated SPA beads) are pre-coated with a polysaccharide sulfate acceptor, such as E. coli. K5 polysaccharide, N-acetyl desulfated heparin, fully desulfated heparin or heparan sulfate. The bead is then incubated with ³⁵[S]-PAPS (a sulfate donor) and an enzyme. Transfer of the radiolabeled sulfate from ³⁵[S]-PAPS onto the sulfate acceptor, which has been bound to the SPA bead, results in the emission of light from the bead. Unused ³⁵[S]-PAPS remains in solution, but is not sufficiently close to the bead to trigger the light emission.

In particular, the present invention encompasses a solid phase assay for N-sulfotransferase activity, and/or N-deacetylase activity of an NDST enzyme including the step of linking a radiolabeled sulfate with a potentially scintillating particulate bound to a polysaccharide sulfate acceptor in the presence of an NDST enzyme, whereby the radiolabeled sulfate transfers to the polysaccharide sulfate acceptor within an activating range of the potentially scintillating particulate to permit emission of a detectable signal.

In the solution phase SPA assay, the reaction occurs in solution in the presence of a free polysaccharide sulfate acceptor, an ³⁵[S]-PAPS sulfate donor and an enzyme. A lectin-coated SPA bead, such as a WGA-SPA bead, is then added to stop the reaction. The sulfate acceptor with or without transferred radiolabeled sulfate from ³⁵[S]-PAPS binds to the WGA-SPA bead, resulting in the emission of light. Unused ³⁵[S]-PAPS remains in solution, but is not sufficiently close to the bead to trigger the light emission.

According to the invention, the solution phase assay for N-sulfotransferase activity, and/or N-deacetylase activity of an NDST enzyme includes the steps of (a) transferring a radiolabeled sulfate from a sulfate donor to a free polysaccharide sulfate acceptor in the presence of an NDST enzyme to form a radiolabeled sulfated polysaccharide, and (b) binding the radiolabeled sulfated polysaccharide to a potentially scintillating particulate, within an activating range of each other to permit emission of a detachable signal.

In a further aspect of the invention, there is provided a scintillating complex for the detection of N-sulfotransferase activity, and/or N-deacetylase activity, the complex formed by the process of associating a radiolabeled sulfate within activating range of a potentially scintillating particulate via a polysaccharide linker to generate a detectable signal indicative of N-sulfotransferase activity, and/or N-deacetylase activity.

The present invention also provides high throughput assays for compounds capable of inhibiting N-sulfotransferase activity, and/or N-deacetylase activity of NDSTs. Moreover, such high throughput assays further provide a means of screening test compounds capable of inhibiting N-deacetylase activity of NDSTs by indirect measurement since enzymatic transfer of the sulfate is a result of both the N-deacetylase and N-sulfotransferase reactions. This will be described in further detail below.

The inventive methods for screening for compounds which inhibit N-sulfotransferase activity, and/or N-deacetylase activity of the NDST enzyme include the step of transferring a radiolabeled sulfate from a sulfate donor to a polysaccharide acceptor in the presence of an NDST enzyme to form a radiolabeled sulfated polysaccharide, the radiolabeled sulfated polysaccharide which when bound to a potentially scintillating particulate within an activating distance emits a detectable signal indicative of N-sulfotransferase activity, and/or N-deacetylase activity. The light emitted in the presence of the test compound may be measured and compared to that emitted in a control assay run in the absence of the test compound.

In one screening method, the transferring step includes transferring the radiolabeled sulfate from a sulfate donor to a free polysaccharide sulfate acceptor to form a radiolabeled sulfate polysaccharide and binding the radiolabeled sulfate polysaccharide to the potentially scintillating particulate. Further encompassed by the present invention is a screening method where the transferring step includes includes transferring the radiolabeled sulfate from a sulfate donor to a sulfate acceptor bound to the potentially scintillating particulate to form the radiolabeled sulfate polysaccharide.

In particular, the invention provides a solution phase method for screening for compounds which inhibit N-sulfotransferase activity, and/or N-deacetylase activity of the NDST enzyme including the steps of: (a) transferring a radiolabeled sulfate from a sulfate donor to a free polysaccharide sulfate acceptor in the presence of an NDST enzyme to form a radiolabeled sulfated polysaccharide, and binding the radiolabeled sulfated polysaccharide to a potentially scintillating particulate, within an activating range of each other to permit emission of a detectable signal; and (b) detecting the light emitted from the particulates in the presence of the test compound to determine N-sulfotransferase, and/or N-deacetylase activity inhibition.

The invention further encompasses a solid phase method for screening for compounds which inhibit N-sulfotransferase activity, and/or N-deacetylase activity of the NDST enzyme including the steps of: (a) linking a radiolabeled sulfate with a potentially scintillating particulate bound to a polysaccharide sulfate acceptor in the presence of an NDST enzyme, whereby the radiolabeled sulfate transfers to the polysaccharide sulfate acceptor within an activating range of the potentially scintillating particulate to permit emission of a detectable signal; and (b) detecting the light emitted from the particulates in the presence of the test compound to determine N-sulfotransferase, and/or N-deacetylase activity inhibition.

The present invention encompasses the use of any molecule capable of serving as a sulfate acceptor by an NDST enzyme. Specifically, the invention encompasses the substitution of any sulfate acceptor referenced herein with a known NDST enzyme substrate, or yet to be identified NDST enzyme substrate, which may include, for example, proteoglycans, polysaccarides, polypeptides, fibroblast growth factor receptors (Development, 126(17):3715-23(1999)), heparin sulfate proteoglycans, glycosaminoglycans, and/or heparan sulfate glycosaminoglycans, among others.

NDSTs are dual catalytic enzymes that catalyze the N-deacetylation of N-acetylglucosamine of glycosaminoglycans followed by N-sulfation of the same sugar. Transfer of the radiolabeled sulfate from ³⁵[S]-PAPS to the N-acetyl sulfate acceptor is the result of the enzymatic reactions of both N-deacetylase and N-sulfotransferase of NDSTs. Thus, inhibitors which inhibit N-sulfotransferase activity of NDSTs could be either inhibitors for N-sulfotransferase of NDSTs or inhibitors for N-deacetylase of NDSTs, or may be inhibitors of both activities.

The invention further encompasses a scintillating complex for the detection of N-sulfotransferase activity, and/or N-deacetylase activity wherein said complex is formed by the process of associating a radiolabeled sulfate within activating range of a potentially scintillating particulate via a polysaccharide linker to generate a detectable signal indicative of N-sulfotransferase activity and/or N-deacetylase activity.

The invention further encompasses a scintillating complex for the detection of N-sulfotransferase activity, and/or N-deacetylase activity, wherein said complex is formed by the process of associating a radiolabeled sulfate within activating range of a potentially scintillating particulate via a polysaccharide linker to generate a detectable signal indicative of N-sulfotransferase activity and/or N-deacetylase activity, wherein the potentially scintillating particulates are coated with wheat germ agglutinin.

The invention further encompasses a scintillating complex for the detection of N-sulfotransferase activity, and/or N-deacetylase activity, wherein said complex is formed by the process of associating a radiolabeled sulfate within activating range of a potentially scintillating particulate via a polysaccharide linker to generate a detectable signal indicative of N-sulfotransferase activity and/or N-deacetylase activity, wherein the detectable signal is light.

The invention further encompasses a scintillating complex for the detection of N-sulfotransferase activity, and/or N-deacetylase activity wherein said complex is formed by the process of associating a radiolabeled sulfate within activating range of a potentially scintillating particulate via a polysaccharide linker to generate a detectable signal indicative of N-sulfotransferase activity and/or N-deacetylase activity, wherein the N-sulfotransferase activity and/or N-deacetylase activity is associated with an NDST enzyme or a derivative thereof.

The invention provides a method of inhibiting N-sulfotransferase activity, and/or N-deacetylase activity of an NDST enzyme, the method including administering to a patient an inhibitory amount of a compound having the following Formula (I) or a pharmaceutically acceptable salt thereof or a prodrug thereof:

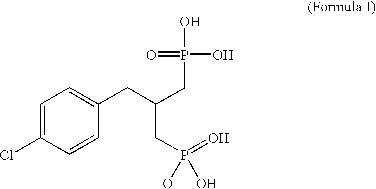

Also encompassed by the invention is a method of inhibiting N-sulfotransferase activity, and/or N-deacetylase activity of an NDST enzyme, the method including administering to a patient an inhibitory amount of a compound having the following Formula (II) or a pharmaceutically acceptable salt thereof or a prodrug thereof:

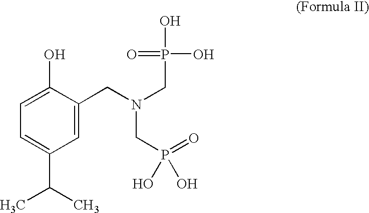

The present invention further provides a method of inhibiting an allergic response, the method including administering to a patient an effective amount of a compound that inhibits the N-sulfotransferase activity, and/or N-deacetylase activity of an NDST enzyme, the compound having Formula (I) shown above or a pharmaceutically acceptable salt thereof or a prodrug thereof.

Moreover, a method of inhibiting an allergic response is provided by the present invention, wherein the method includes administering to a patient an effective amount of a compound which inhibits the N-sulfotransferase activity, and/or N-deacetylase activity of an NDST enzyme, the compound having Formula (II) shown above or a pharmaceutically acceptable salt thereof or a prodrug thereof.

### BRIEF DESCRIPTION OF THE DRAWINGS

FIG. 1A is a schematic representation of the solid phase SPA assay for N-sulfotransferase activity of the present invention where WGA-SPA beads coated with desulfated heparin are incubated with NDST and the radiolabeled ³⁵[S]-PAPS.

FIG. 1B is a schematic representation of the solution phase SPA assay for N-sulfotransferase activity of the present invention where non-coated WGA-SPA beads are added following the N-sulfotransferase reaction.

FIG. 2 shows a comparison between the solid phase and solution phase SPA assays with respect to the signal generated at increasing concentrations of the recombinant human sulfotransferase domain of NDST-2 (ST2).

FIG. 3 depicts the dependence of N-sulfotransferase activity on the concentration of ³⁵[S]-PAPS in the solid phase SPA assay where experimental data was obtained using recombinant human sulfotransferase domain of NDST-2 (ST2) and control data was obtained in the absence of ST2.

FIG. 4 shows the time dependence of ³⁵[S]-PAPS incorporation into fully desulfated heparin during the solid phase SPA assay where experimental data was obtained in the presence of recombinant human sulfotransferase domain of NDST-2 (ST2) and control data was obtained in the absence of ST2.

FIG. 5 shows the N-sulfotransferase activity of the recombinant human sulfotransferase domain of NDST-2 (ST2) as presented in a double-reciprocal plot for the determination of the Km value of PAPS using the solid phase SPA assay according to the present invention.

FIG. 6A shows the identification of Compound A as an inhibitor of N-sulfotransferase activity by the solid phase SPA assay using the recombinant human sulfotransferase domain of NDST-2 (ST2).

FIG. 6B shows the identification of Compound B as an inhibitor of N-sulfotransferase activity by the solid phase SPA assay using the recombinant human sulfotransferase domain of NDST-2 (ST2).

FIG. 6C shows the identification of Compound A as an inhibitor of N-sulfotransferase activity by the solid phase SPA assay using the recombinant human wild-type NDST-2.

FIG. 6D shows the identification of Compound B an inhibitor of N-sulfotransferase activity by the solid phase SPA assay using the recombinant human wild-type NDST-2.

FIG. 7 shows the inhibitory effects of Compound A and Compound B on N-sulfotransferase activity by a manual sulfotransferase assay using the recombinant human NDST-2 (wild-type). The percent inhibition for Compound A at final concentration of 6.25 μM was 94.7% and the percent inhibition for Compound B at final concentration of 6.25 μM was 64.8%.

### DETAILED DESCRIPTION OF THE INVENTION

The term “potentially scintillating particulate”, “scintillating particulate” and the like as used herein refer to lectin-coated scintillation proximity assay structures (e.g. beads). Lectins are polyvalent proteins that agglutinate cells and bind complex carbohydrates (Sharon, N. and Lis, M., Editors; (1989) Lectins. Chapman and Hall, New York, N.Y.). Many lectins, in particular those having a high affinity for N-acetylglucosamine, are capable of binding to the polysaccharide sulfate acceptor. In one desired embodiment, wheat germ agglutin is the substance to be pre-coated on the beads. Wheat germ agglutinin (WGA) is a lectin isolated from Triticum vulgaris (Triticum vulgaris lectin). Other suitable lectins are Datura stramonium lectin (DSL), Lycopersicon esculentum agglutinin (LEA), Phytolacca americana mitogen (Pokeweed mitogen, PWM) and Solanum tuberosum agglutinin (STA).

The term “prodrug” as used herein means compounds that are drug precursors which, following administration to a patient, release the drug in vivo via some chemical or physiological processes.

Sulfotransferases are enzymes which are involved in the transfer of sulfate from adenosine 3′ phosphate phosphosulfate (PAPS) to specific substrates, such as growing proteoglycans (Horwitz et al., J. Cell Biol. 38: 358, 1968; Young et al., J. Cell Biol. 57: 175, 1973). In one specific example of a sulfotransferase-mediated reaction, the biosynthesis of sulfated proteoglycans, such as the glycosaminoglycans, heparin and heparan sulfate, occurs in a precise, step-wise manner. Once the polysaccharide polymers have been formed, they undergo a series of modification reactions. The first of these reactions include the N-deacetylation and N-sulfation of glucosamine. It is known that the N-deacetylation of N-acetyl-D-glucosamine residues is required for the N-sulfation reaction to occur and these two reactions appear to be tightly coupled. The quantity and distribution of NDST appears to determine not only the extent of the respective modification, but also the extent of subsequent modifications. Therefore, an assay for N-sulfotransferase activity according to the present invention is indirectly an assay for N-deacetylase activity. Likewise, an inhibitor of N-sulfotransferase activity may be an inhibitor of N-acetylase, an inhibitor of N-sulfotransferase, or an inhibitor of both reactions. This will be discussed in further detail below.

A large advantage of a scintillation proximity assay is that it does not require separation of bound molecular species from free species. Such an assay minimizes the need to handle potentially hazardous/radioactive substances and is more amenable to automation.

In accordance with the assays for N-sulfotransferase activity of NDSTs of the present invention, particles in the form of beads or other structures are impregnated and/or coated with a material capable of fluorescence when excited by the radioactive energy. In one desired embodiment, the beads are pre-coated with wheat germ agglutinin (WGA). Wheat germ agglutinin is capable of binding to glycoproteins such as N-glycosaminoglycans that are substrates for glucosaminyl N-deacethylase/N-sulfotransferases (NDSTs). The enzyme under investigation allows ³⁵[S]-sulfate to be brought into proximity with a potentially scintillating particulate (bead). In particular, the ³⁵[S]-sulfate is transferred from a sulfate donor (³⁵[S]-adenosine 3′-phosphate 5′-phosphosulfate (PAPS)) to a sulfate acceptor (e.g. fully desulfated polysaccharide) to form a radiolabeled sulfated polysaccharide which when bound to the particulate via WGA, or another suitable lectin, generates a detectable light signal. By placing the radiolabeled species in close proximity to the scintillant-containing particulate, the scintillant is activated, causing emission of light, which can be detected conventionally using a scintillation counter. The amount of light produced is directly proportional to the amount of enzyme present in the sample. Additionally, other monitoring devices such as photomultiplier tubes may be utilized in the measurement.

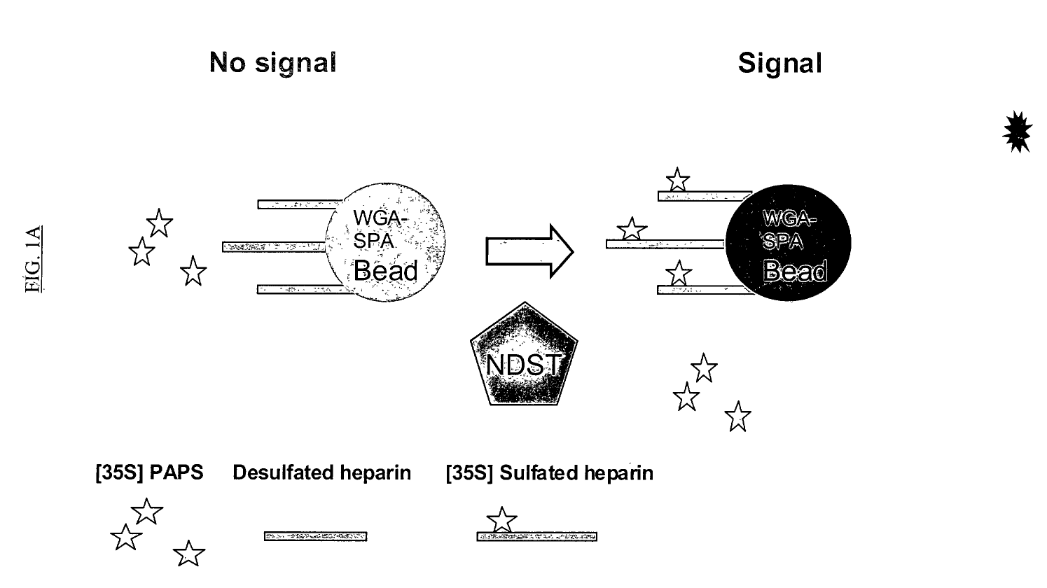

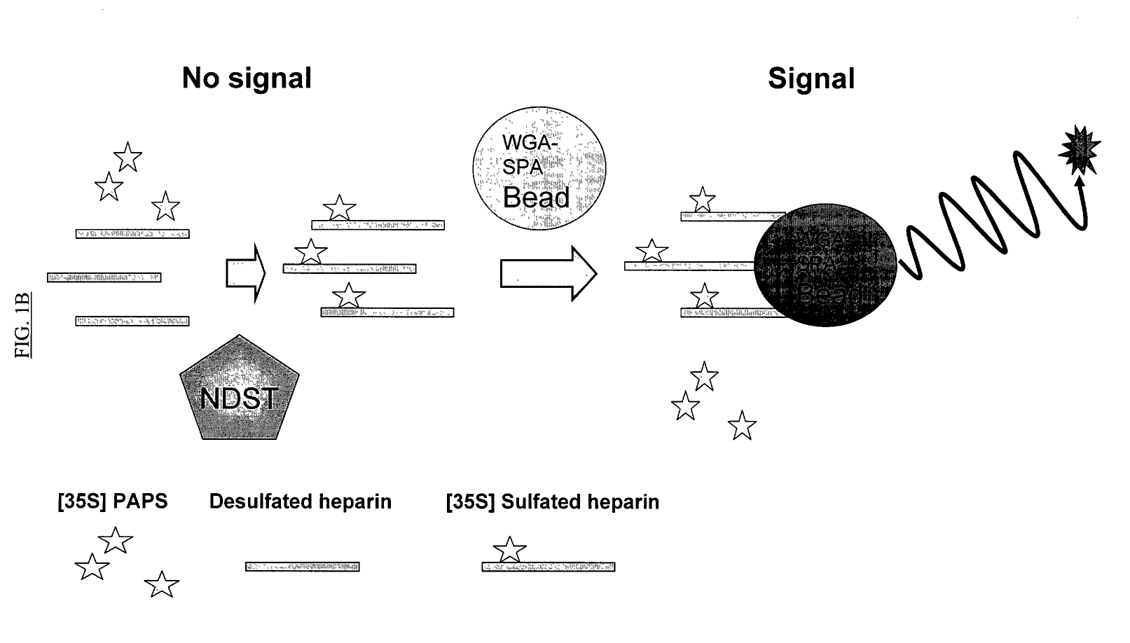

Referring now to FIG. 1A, in one embodiment of the invention, a solid phase assay for N-sulfotransferase activity of an NDST enzyme is provided, the assay including the step of linking an ³⁵[S]-labeled sulfate with a potentially scintillating particulate bound to a polysaccharide sulfate acceptor in the presence of an NDST enzyme and a sulfate donor under suitable conditions to allow the enzymatic transfer of a labeled sulfate from the sulfate donor to the bound sulfate acceptor, each of the scintillating particulates emitting detectable light pulses upon activation from the transferred sulfate, the light pulses being correlative to the N-sulfotransferase activity of NDST enzyme. In one embodiment of the solid phase assay, the particulates are coated with wheat germ agglutinin which is capable of binding to glycoproteins. In particular, wheat germ agglutinin-coated SPA particulates may be covalently bound to a desulfated glycosaminoglycan, such as fully desulfated heparin. In the solid phase assay, these pre-coated particulates are combined with NDST enzyme in the presence of the sulfate donor (³⁵[S]-adenosine 3′-phosphate 5′-phosphosulfate) to give rise to sulfated glycosaminoglycan on the beads, which yields a scintillation signal that is a measure of enzymatic activity. It is the enzyme under investigation which allows the ³⁵[S]-sulfate to be brought into proximity with the beads to generate the detectable signal.

In the solid phase assay, the sulfate acceptor may be a glycosaminoglycan or a modified version thereof. In particular, the sulfate acceptor may be selected from the group consisting of: E. coli K5 polysaccharide, N-acetyl desulfated heparin, fully desulfated heparin and heparan sulfate.

With reference now to FIG. 1B, the invention further provides a solution phase assay for N-sulfotransferase activity of an NDST enzyme, the assay including the steps of (a) transferring an ³⁵[S]-labeled sulfate from a sulfate donor to a free polysaccharide sulfate acceptor (e.g. fully desulfated heparin) in the presence of an NDST enzyme, so as to form a radiolabeled sulfated polysaccharide, and (b) binding the radiolabeled sulfated polysaccharide to a potentially scintillating particulate within an activating range of each other to permit emission of a detectable signal. In particular, the scintillating particulates emit detectable light pulses upon activation from the transferred sulfate, the light pulses being correlative to the N-sulfotransferase activity of NDST enzyme. In this assay, wheat germ agglutinin-coated SPA particulates (beads) bind to the radiolabeled sulfated polysaccharide that has been produced as a result of the N-sulfotransferase activity. Once this radiolabeled sulfated polysaccharide binds to the beads, the radiolabeled sulfate is brought into close enough proximity to the beads to facilitate the generation of detectable light.

With respect to the solution phase assay, it is well within the contemplation of the present invention that each of the components of the assay may be added simultaneously. However, the efficiency with which the scintillating complex may be formed may be less than that obtained by the format shown in FIG. 1B.

In one embodiment of the solution phase assay, the sulfate donor is ³⁵[S]-adenosine 3′-phosphate 5′-phosphosulfate (PAPS). In a further embodiment, the sulfate acceptor is a glycosaminoglycan or a modified version thereof (e.g. a fully desulfated version). In particular, the sulfate acceptor may be selected from E. coli K5 polysaccharide, N-acetyl desulfated heparin, fully desulfated heparin and heparan sulfate.

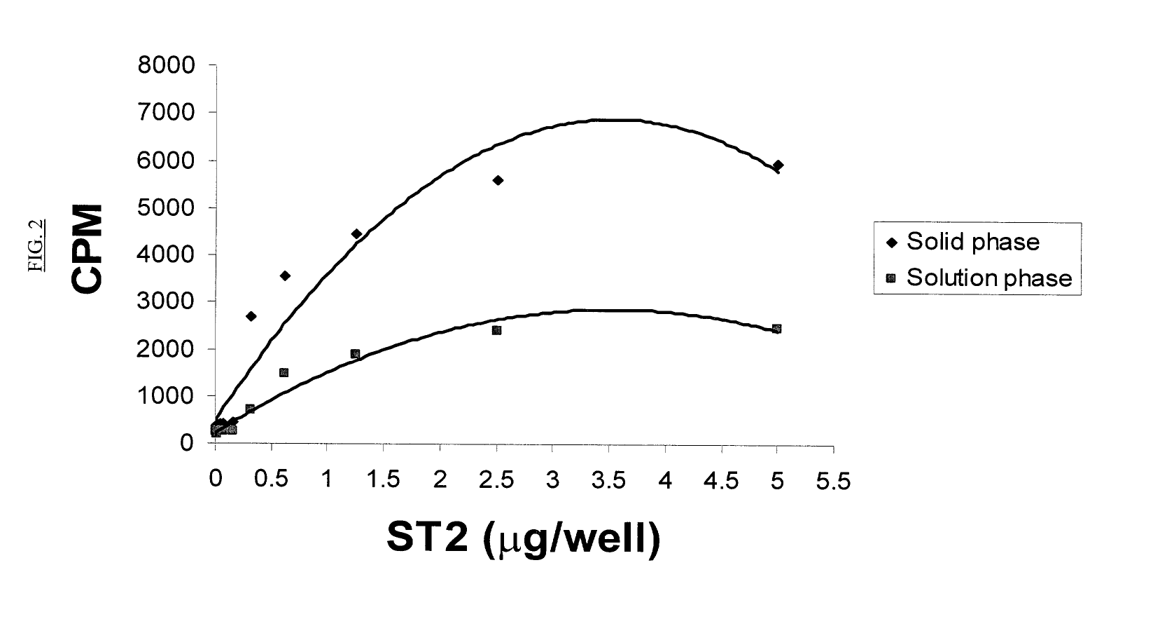

We refer now to FIG. 2, which shows the comparison between a solid phase and solution phase SPA assays of the present invention. In the solid phase assays, fully-desulfated heparin that had been immobilized onto the WGA-SPA beads corresponded to the sulfate acceptor. In the solution phase assay, unbound fully-desulfated heparin corresponded to the sulfate acceptor. The sulfate donor for both the solid phase and solution phase assays was ³⁵[S]-PAPS. The enzyme being investigated corresponded to recombinant human sulfotransferase domain of NDST-2 (ST2). Sulfotransferase activity was measured at different time points throughout a five-hour assay. The results shown indicate that at increasing concentrations of ST2, (up to 2.5 μg/well), an increasing signal correlating to sulfotransferase activity was observed for both the solid phase and solution phase SPA assays. In the solid phase assay, the average CPM at the concentration of 2.5 μg/well ST2 was 5588 (signal) while the average CPM in the absence of enzyme is 348 (noise); the signal-to-noise ratio was about 16:1. In the solution phase assay, the average CPM at the concentration of 2.5 μg/well ST2 was 2395, while the average CPM in the absence of enzyme was 240; the signal-to-noise ratio was about 10:1.

The invention further provides a scintillating complex for the detection of N-sulfotransferase activity. The complex is formed by the process of associating a radiolabeled sulfate within an activating range of a potentially scintillating particulate via a polysaccharide linker to generate a detectable signal indicative of N-sulfotransferase activity. In particular, the potentially scintillating particulates are coated with a lectin, such as wheat germ agglutinin, which is capable of bonding to the polysaccharide linker. In one embodiment, the polysaccharide linker is a glycosaminoglycan or a modified version thereof. In particular, the sulfate acceptor which serves as the polysaccharide linker may be selected from the following: E. coli K5 polysaccharide, N-acetyl desulfated heparin, fully desulfated heparin and heparan sulfate. As described above, the radiolabeled sulfate is derived from a sulfate donor (PAPS). In one embodiment, the detectable signal indicative of N-sulfotransferase activity of an NDST enzyme or a derivative thereof, is light. This light may be detectable by use of a scintillation counter or photo-multiplier tubes.

The present invention is useful in conducting enzyme kinetic studies with NDSTs. As described above, it is the enzyme which allows the radiolabel from the substrate (³⁵[S]-PAPS) to be brought in close enough proximity to the beads to cause them to emit light energy. All of the assay components are present in the same receptacle, such as a microtiter well or vial. Kinetic experiments may be carried out by measuring the light energy emitted from a given receptacle at various time intervals to determine the reaction rate of the enzyme.

The assay advantageously employs a sulfate donor (radiolabeled PAPS) which is conveniently available from a commercial source. Moreover, the enzyme specificity for the sulfate donor is not affected by the radiolabeling.

The NDST enzyme under investigation may include any member of the NDST superfamily of enzymes. In particular, NDST enzyme may be selected from the following: NDST-1, NDST-2, NDST-3, NDST-4, fragments, derivatives, and mutated versions thereof. For example, the invention may be useful to assay for biologically active polypeptide fragments or analogs of a recombinantly produced NDST enzyme. By biologically active it is meant possessing any in vivo or in vitro activity which is characteristic of the native NDST enzyme. A polypeptide fragment possessing at least 10%, preferably at least 40%, or most preferably at least 90% of the activity of wild-type NDST enzyme in any in vivo or in vitro sulfotransferase assays is considered biologically active and would be useful in the invention. The term “fragment” as applied to the polypeptide would ordinarily be at least about 20 contiguous amino acids, preferably at least about 40 contiguous amino acids, and most preferably at least about 60-80 contiguous amino acids in length. Fragments can be generated by methods known to those skilled in the art.

In preferred embodiments, the NDST enzyme under investigation is from a mammalian source, such as from a human. GenBank Accession Numbers corresponding to the human cDNA sequence and predicted protein sequence are as follows: NDST-1 (SWISS-PROT Accession No:U36600); NDST-2 (SWISS-PROT Accession No:U36601); NDST-3 (Genbank Accession No:AF074924); and NDST-4 (Genbank Accession No:AB036429).

Desirably, a derivative or mutated version of an NDST would exhibit at least 70%, preferably at least 80%, more preferably at least 90%, and most preferably at least 95% or even at least 99% homology with all or part of a naturally occurring NDST enzyme. Differences in amino acid sequence may be by conservative amino acid substitutions, for example, substitution of one amino acid for another of the same class (e.g. arginine for lycine: valine for isoleucine, etc.) or alternatively by one or more non-conservative amino acid substitutions, deletions, or insertions which do not destroy the analog's biological activity as measured by in vivo or in vitro assays for the NDST enzyme. Modifications of the enzyme or its fragments may include chemical derivativation of polypeptides, such as acetylation, or carboxylation. Also included are modifications of glycosylation or phosphorylation. Moreover, the enzyme or biologically active fragments thereof may be modified for the purpose of increasing peptide stability.

As described above, the inventive assays of the present invention may include the step of measuring the light emitted from the particulates attributable to the N-sulfotransferase activity of the NDST. The assays of the present invention may further include the step of determining the amount of N-sulfotransferase activity of the NDST based on this measurement. The amount of N-sulfotransferase activity may be determined as compared to a control sample containing a known amount of such activity.

The ³⁵[S]-isotope of the radiolabel has a relatively low energy beta-emission. Consequently, only that portion of the labeled PAPS which is enzymatically transferred to the sulfate acceptor and is brought in close proximity to the scintillant coated on or embedded in the beads will result in scintillation events that can be counted. Labeled ³⁵[S]-PAPS that is not acted upon by the enzyme so as to be brought within activating range of the potentially scintillating particulate will be at too great a distance from the scintillant surface to produce scintillations. Therefore, beta-decay energy would be dissipated in a liquid aqueous medium for these uncomplexed species.

As noted above, and in accordance with the present invention, a scintillant substance is integrated within the beads or is coated on the beads to give off light energy when radiolabeled PAPS is brought in close enough proximity to the scintillant substance to cause excitation thereof, i.e. by enzymatic transfer of the radioactive sulfate to the sulfate acceptor bound indirectly to the bead surface via a suitable lectin. Various types of scintillant substances may be used. However, since the assay takes place in aqueous solution, the substance is desirably insoluble in water so that it does not disassociate from the beads during the assay procedure. Moreover, the substance, e.g. fluorescer, employed must be excitable to a higher energy state by the particular wavelength that is associated with the energy rays emitted from the radiolabeled PAPS. Furthermore, the scintillant substance desirably releases a specific amount of light energy when returning to its normal energy state so that it may be detected by a scintillation counter or other detection device such as a photomultiplier tube.

The scintillant substance can include aromatic hydrocarbons such as p-terphenyl, p-quaterphenyl and their derivatives, as well as derivatives of the oxazoles and 1,3,4-oxadiazoles, such as 2-(4-2-butyl phenyl)-5-(4-biphenylyl)-1,3,4-oxadiazole and 2,5-diphenyloxazole. Furthermore, the particulates may include a wave-length shifter such as 1,4-bi(5-phenyl-2-oxazolyl)-benzene, 9,10-diphenylanthracene, 1,4-bis(2-methylstyryl)-benzene and others. The function of the wavelength shifter would be to absorb the light emitted by the scintillant substance and re-emit longer wavelength light, which would be more suitable to the photo-sensitive detectors used in scintillation counters.

The scintillant substances can be incorporated into the particulates by a variety of methods. For example, the particulates may be manufactured of a polymeric substance. The scintillators may be dissolved into the monomer mix prior to polymerization, so that they are evenly distributed throughout the resultant polymeric particulates. Alternatively, the scintillant substances may be dissolved in a solution of the polymer used to form a particulate and the solvent removed to leave a homogenous mixture.

Various types of beads may be utilized such as including, but not limited to, polyacrylamide, acrylamide, agarose, polystyrene, polypropylene, polycarbonate or Sepharose-4B beads (Pharmacia Fine Chemicals, Uppsala, Sweden). The invention may be carried out with shapes other than bead-like structures. Moreover, other types of support structures may be used provided that they may be coated with wheat germ agglutinin and a fluorescer may be integrated therewith or thereon. Sepharose-4B beads are commercially available in an activated state. For example, compounds such as cyanogen bromide are incorporated in the beads to covalently bind with certain ligands, such as wheat germ agglutinin. Wheat germ agglutinin may be bound to the beads by placing the beads in a solution containing wheat germ agglutinin and an appropriate buffer. Thereafter, excess wheat germ agglutinin is washed away and the remaining active sites on the beads to which no wheat germ agglutinin had attached are blocked with an appropriate blocking agent, such as glycine. It is noted that scintillation proximity assay beads coated with wheat germ agglutinin are also available commercially (Amersham Pharmacia Biotech).

It is noted that suitable particulates for scintillation proximity assays include fiber mats which incorporate a fluorescer, such as those described in European Patent Application No. 0378059. In one format, the fiber mat consists of solid scintillant forming a matrix. The scintillant fiber may be composed of a scintillant polymer, such as polyvinyltoluene. Alternatively, an organic scintillant such as 2,5-diphenyloxazole (PPO) or anthracene may be coated onto a fiber mat which is made from non-scintillant material. The advantage of the fiber mesh format is that it presents a large surface area upon which binding reactions can occur.

In another aspect of the invention there are provided methods for screening for compounds which inhibit N-sulfotransferase activity of the NDST enzyme that include the steps of: (a) transferring a radiolabeled sulfate from a sulfate donor to a polysaccharide sulfate acceptor in the presence of an NDST enzyme to form a radiolabeled sulfated polysaccharide, the radiolabeled sulfated polysaccharide which when bound to a potentially scintillating particulate within activating distance emits a detectable signal indicative of N-sulfotransferase activity; and (b) detecting the light emitted from the particulates in the presence of the test compound to determine N-sulfotransferase inhibition. In one embodiment, the detecting step includes measuring and comparing the light emitted in the presence of the test compound relative to a control assay run in the absence of the test compound. The light emitted is desirably measured by scintillation counting. However, it is well within the contemplation of the present invention that the measuring may be performed by other suitable methods, such as the use of photomultiplier tubes. In one embodiment, the measuring is performed at different time intervals so as to obtain a reaction rate.

In one embodiment of the inventive method for screening for inhibitory compounds, the sulfate donor corresponds to ³⁵[S]-adenosine 3′-phosphate 5′-phosphosulfate (PAPS). In another embodiment, the sulfate acceptor is a glycosaminoglycan or a modified version thereof. In particular, the sulfate acceptor may be selected from the following: E. coli K5 polysaccharide, N-acetyl desulfated heparin, fully desulfated heparin and heparan sulfate.

Potentially scintillating particulates for use in the screening method are coated with a lectin, such as wheat germ agglutinin, the lectin being capable of binding to the polysaccharide sulfate acceptor, both in the presence and absence of the transferred sulfate.

In one desired embodiment of the screening method, the transferring step includes transferring the radiolabeled sulfate from a sulfate donor to a free polysaccharide sulfate acceptor to form a radiolabeled sulfate polysaccharide and binding the radiolabeled sulfate polysaccharide to the potentially scintillating particulate. For example, the invention provides a solution phase method for screening for compounds which inhibit N-sulfotransferase activity of the NDST enzyme that includes the step of transferring a radiolabeled sulfate from a sulfate donor to a free polysaccharide sulfate acceptor in the presence of an NDST enzyme to form a radiolabeled sulfated polysaccharide, and binding the radiolabeled sulfated polysaccharide to a potentially scintillating particulate, within an activating range of each other to permit emission of a detectable signal. The solution phase method further includes the step of detecting the light emitted from the particulates in the presence of the test compound to determine N-sulfotransferase inhibition. Suitable sulfate donors and sulfate acceptors for the solution phase screening method are the same as those described above.

In a further desired embodiment of the screening method described above, the transferring step includes transferring the radiolabeled sulfate from a sulfate donor to a sulfate acceptor bound to the potentially scintillating particulate to form the radiolabeled sulfate polysacchride. For example, a solid phase method for screening for compounds which inhibit N-sulfotransferase activity of the NDST enzyme is provided by the present invention. The solid phase method includes the step of linking a radiolabeled sulfate with a potentially scintillating particulate bound to a polysaccharide sulfate acceptor in the presence of an NDST enzyme, whereby the radiolabeled sulfate transfers to the polysaccharide sulfate acceptor within an activating range of the potentially scintillating particulate to permit emission of a detectable signal. The solid phase method further includes the step of detecting light emitted from the particulates in the presence of the test compound to determine N-sulfotransferase inhibition. Suitable sulfate donors and sulfate acceptors for the solid phase screening method are the same as those described above.

It is noted that a compound “inhibits” N-sulfotransferase activity of NDST if the level of transfer of sulfate from PAPS to a specific substrate, such as glycosaminoglycan, is decreased as compared with the level of sulfate transfer in the absence of a test compound. In one embodiment, the compound inhibits N-sulfotransferase activity by 50% or more as compared to a control sample not contacted with the test compound.

It is further noted that the test compound for screening may include both biologic agents and chemical compounds. Biologic agents include, but are not limited to, nucleic acid sequences, peptides, and polypeptides. The test compound may optionally be a combinatorial library for screening a plurality of compositions. Compounds which are identified in the method of the invention can be further evaluated by other suitable methods. For example, if the combinatorial library includes a biologic agent such as a DNA sequence, it may be cloned and sequenced either in solution or after binding to a solid phase by any method usually applied to the detection of a specific DNA sequence. Combinatorial chemistry methods are included in the screening method of the invention for identifying chemical compounds that inhibit the N-sulfotransferase activity of NDST.

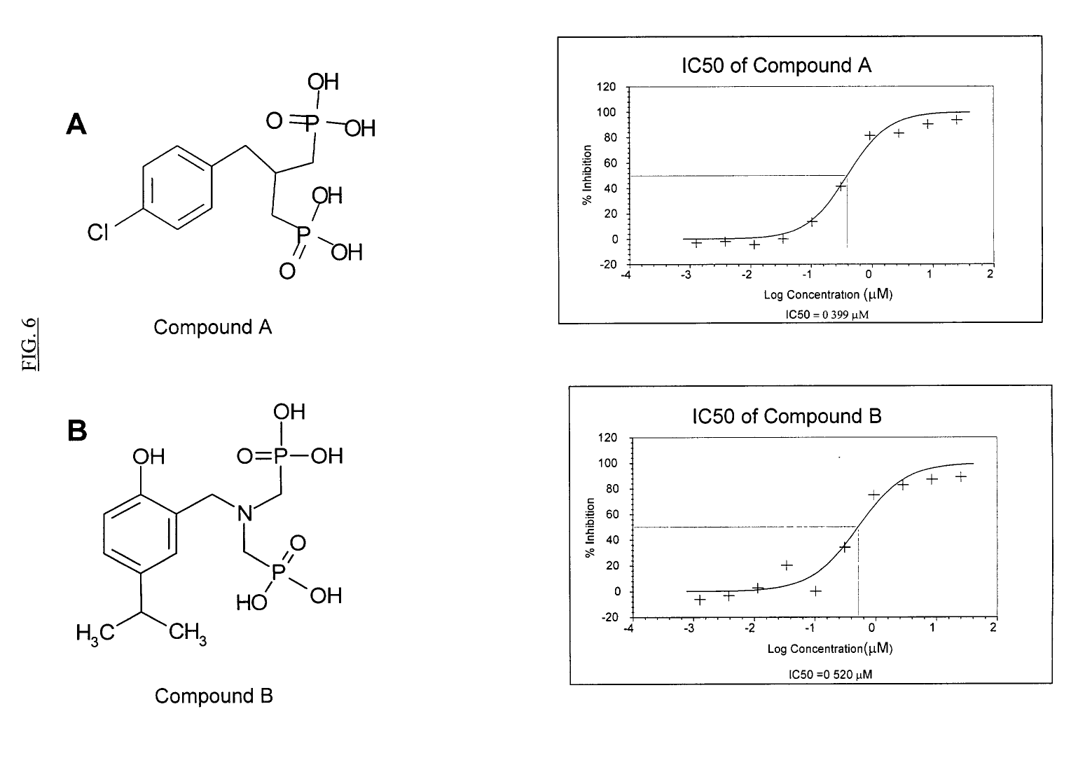

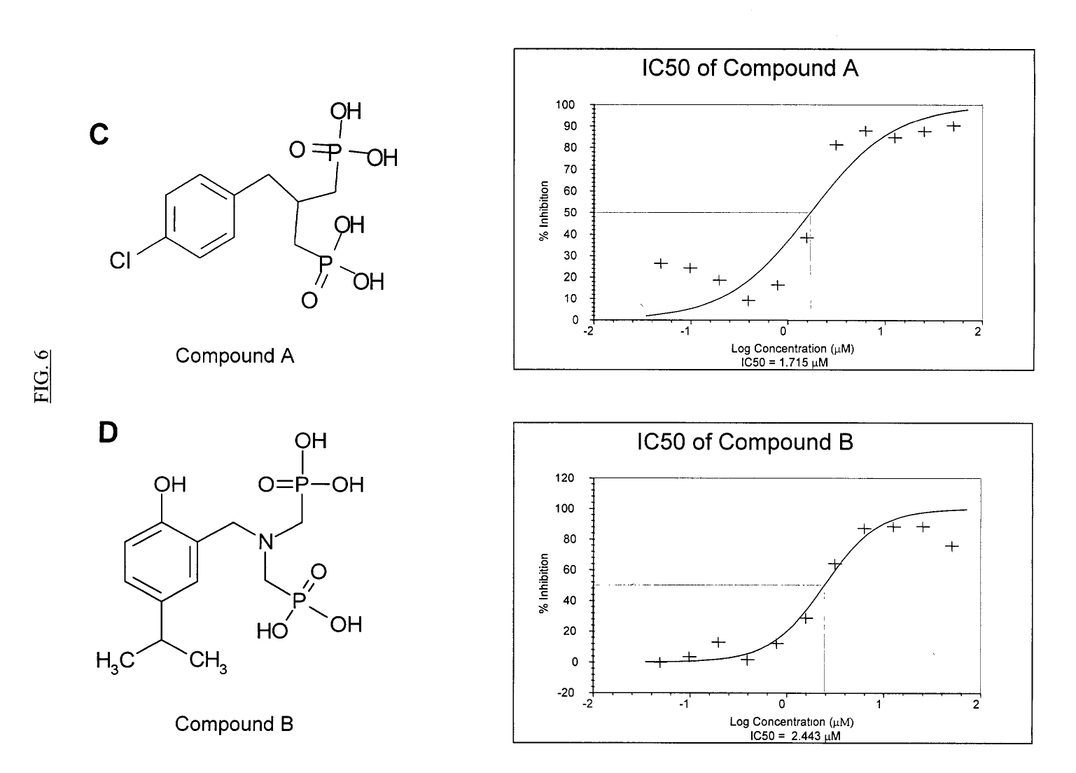

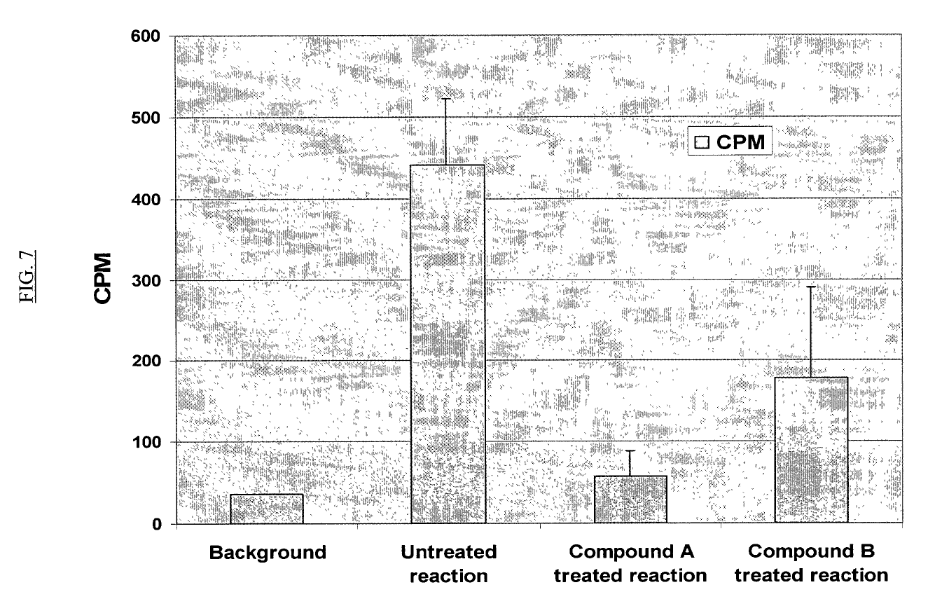

Using the solid phase SPA assay provided by the present invention described above, two compounds were determined to be inhibitors of N-sulfotransferase activity using the recombinant human sulfotransferase domain of NDST-2 (ST2) or recombinant human wild-type NDST-2, as shown in FIGS. 6(A-D). The results in FIG. 6 indicate that the IC₅₀ of Compound A is about 0.399 μM (ST2) and 1.715 μM (wild-type). The IC₅₀ of Compound B is about 0.520 μM (ST2) and 2.443 μM (wild-type). The results suggest that both Compound A (Formula I) and Compound B (Formula II) are N-sulfotransferase inhibitors of NDST-2.

Compound A (Formula I) may be prepared by the general procedures described by Nguyen Mong Lan, et al. in European Patent Application Publication No. EP 173,041 A1, published Mar. 5, 1986; and more generally in European Patent Application Publication No. EP 173,041 B1, published Nov. 30, 1988, which are hereby incorporated by reference herein in their entirety.

Compound B (Formula II) may be prepared by the general procedures described by W. Chen and Y. Xie in Xaoxue Xuebao (1984), 19(11) 865-8; and more generally by X. Xu et al. in Zhongguo Yaolixue Yu Dulixue Zazhi (1987), 1(5) 386-7, which are hereby incorporated by reference herein in their entirety.

Since processing of the biosynthesis of heparin and heparan sulfate is dependent on N-sulfation, the NDSTs that catalyse this initial reaction are important in the determination of the final structures of heparin and heparan sulfate and production of the biological activities [Sasisekharan, R. and Venkataraman, G. (2000) Curr. Opin. Chem. Biol. 4, 626-631]. It is noted that inhibitors of NDSTs are likely to be useful to treat a number of disorders.

As described above, NDST-2 is a rate-limiting enzyme of heparin modification in mast cells and is required for granular formation. Mast cells from NDST-2-deficient mice lack normal granules and contain significantly less histamine and proteases. Therefore, inhibitors of NDST-2 are likely to be useful for the treatment of asthma and other allergic diseases including allergic rhinitis, extrinsic allergic alveolitis, pulmonary eosinophilia, anaphylaxis, food allergy, urticaria and angioedema, otitis media, ocular allergy, atopic and contact dermatitis, insect-sting allergy, and drug reactions.

Moreover, mast cells also release a number of immunoregulatory cytokines such as interleukin-1 (IL-1), IL-3, IL-4, IL-5, IL-6, IL-8, IL-13, tumor-necrosis factor-alpha (TNF-alpha), interferon-gamma and granulocyte-macrophage colony-stimulating factor (GM-CSF) and lipid-derived inflammatory mediators, such as leukotrienes and prostaglandins [Metcalfe, D. D. et al. (1997) Physiol. Rev. 77,1033-1079]. These molecules, as well as histamine and mast cell proteases, have been implicated in the pathogenesis of a variety of clinical conditions associated with local inflammation and tissue remodeling. The application of NDST-2 inhibitors is also devoted to the mast cell-associated inflammatory diseases, including rheumatoid arthritis, chronic obstructive pulmonary disease (COPD), fibrotic lung disease, neurofibromatosis, psoriasis, scleroderma, interstitial cystitis, ulcerative colitis, and Crohn's disease.

The biosynthesis of sulfated heparan sulfate requires enzymes NDST-1, NDST-3 or NDST-4. The sulfated heparan sulfate proteoglycans are abundant cell-surface molecules that function as co-receptors in the formation of receptor-signaling complexes. [Bernfield, M. et al. (1999) Annu. Rev. Biochem. 68,729-777]. It has been demonstrated that cell surface heparan sulfate proteoglycan can immobilize basic fibroblast growth factors (bFGF), increase its local concentration, change its conformation, and present it to its signaling receptors [Yayon A. et al. (1991) Cell 64,841-848]. Reduction of the sulfation of the heparan sulfate reduces binding of bFGF to its cell surface receptor, suggesting the sulfated heparan sulfate is required for bFGF function [Rapraeger, A. C. et al. (1991) Science 252,1705-1708]. The co-receptor role is also applied for the binding of microbial pathogens to host cells [Feyzi, E. et al. (1997) J. Biol. Chem. 272,24850-24857] and the binding of cytokines and chemokines to receptors [Hoogewerf, A. J. (1997) Biochemistry 36,13570-13578]. In addition, heparan sulfate proteoglycans play important roles in cellular adhesion, migration, proliferation, differentiation, and angiogenesis. Inhibitors of NDST-1, NDST-3 or NDST-4 are likely to be useful for the treatment of microbial infections, wound repair, inflammatory diseases, and certain neoplastic conditions.

The present invention provides a method of inhibiting N-sulfotransferase activity, or alternatively, or in addition to N-deacetylase activity, of an NDST enzyme, the method including administering to a patient an inhibitory amount of a compound having Formula (I) shown below or a pharmaceutically acceptable salt thereof or a prodrug thereof:

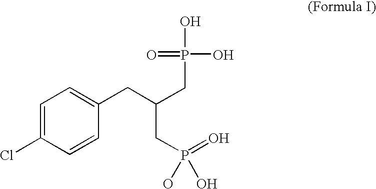

The invention further provides a method of inhibiting N-sulfotransferase activity, or alternatively, or in addition to N-deacetylase activity, of an NDST enzyme, the method including administering to a patient an inhibitory amount of a compound having Formula (II) below or a pharmaceutically acceptable salt thereof or prodrug thereof:

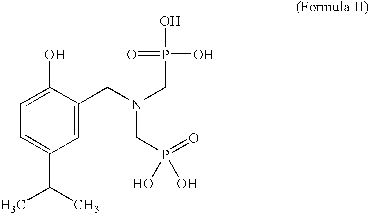

Also encompassed by the invention is a method of inhibiting an allergic response, the method including administering to a patient an effective amount of a compound which inhibits the N-sulfotransferase activity of an NDST enzyme, the compound having (Formula I) shown above.

Further provided by the invention is a method of inhibiting an allergic response, the method including administering a patient an effective amount of a compound which inhibits the N-sulfotranferase activity of an NDST enzyme, the compound having (Formula II) shown above.

In one application, compounds according to Formula (I) or Formula (II), or other test compounds which have been determined to inhibit N-sulfotransferase activity, N-deacetylase activity, or both activities will be admixed with a pharmaceutically acceptable carrier substance, e.g., physiological saline, and administered to a mammal, e.g., a human suffering from an allergic disorder. The particular mode of administration (e.g., intravenously, intramuscularly, orally, parenterally, or transdermally) will depend upon the particular condition being treated and the general status of the mammal. The dosage of the compound will also vary, depending on such factors as the type and severity of the disease, but will generally be a dose sufficient to inhibit the transfer of sulfate from PAPS to the specific substrates, such as growing proteoglycans.

### EXAMPLES

### Example 1

### Preparation of Recombinant Human NDST-2

Cloning of human NDST-2 cDNA and a pFASTBac-GST-NDST-2 construct: To clone human NDST-2, the DNA fragment consisting of nucleotides 133-2651 of the human NDST-2 cDNA listed under Genbank Accession No. U36601 and Gl No. gi/1036798 was amplified by PCR utilizing HUVEC cDNA as a template, and oligonucleotide primers based upon the cDNA sequence of human NDST-2. The human NDST-2 gene is published in Karlinsky, J. B. et al. (1998) Biochem. J. 332, 303-307. The forward primer was flanked with a BamHI on the 5′ end of the gene 5′-ATCGGATCCTATTATGTGTCCACCAGCCCTAAG-3′ (SEQ ID NO:1) and the reverse primer was flanked with an XbaI on the 3′ end of the gene 5′-ACCTCTAGACATCAGCCCAGACTGGAATGCTG-3′ (SEQ ID NO:2). PCR was performed using the eLONGase Amplification system (Life Technologies, Gaithersburg, Md.) in a total volume of 50 μl. The PCR mixture was incubated at 94° C. for 30 sec, 55° C. for 30 sec, and 68° C. for 3 min, and this was repeated for 35 cycles. The PCR products were separated by gel electrophoresis on a 1% agarose gel, and the DNA was visualized by ethidium bromide staining. A 2536-bp DNA fragment corresponding to the NDST-2 was purified from the agarose gel using the QIAGEN gel extraction kit (QIAGEN, Valencia, Calif.), and then digested using BamHI and XbaI restriction endonucleases and directionally cloned into pFASTBac-GST vector (Life Technologies) using 5 U/μl T4 DNA ligase (Life Technologies). The ligation mixture was used to transfect DH5 alpha competent E. coli cells (Life Technologies), and transfected cells were plated onto Lauria-Bertani (LB) plates containing 50 μg/ml ampicillin. Plates were incubated overnight at 37° C., and colonies were isolated and grown overnight at 37° C. in LB broth containing 50 μg/ml ampicillin. Plasmids were isolated using the QIAGEN miniprep spin kit, resuspended in 50 μl distilled water, and sequenced using an ABI cycle sequencer (PE Biosystems, Foster City, Calif.).

A-FLAG-NDST-2 fusion construct: Plasmid containing the coding sequence of the protein A-FLAG-NDST-2 fusion gene was generated by subcloning of NDST-2 from pFASTBac-GST-NDST-2 construct. NDST-2 was subcloned into the pTV5 vector in three steps. The first step was to amplify, by PCR, the 5′ portion of the NDST-2 gene from pFASTBac-GST with a flanking XhoI and a FLAG tag on the 5′ of the gene and an XbaI site on the 3′ end of the gene (the forward primer 5′GGAAAACTCGAGATGGATTACAAGGATGATGATGATAAGAAGGCCAAG GAACCCTTGCCC-3′ (SEQ ID NO:3) and the reverse primer 5′-GGAAAATCTAGAACGAAAACGACTAGACTCCAG-3′ (SEQ ID NO:4). The PCR amplicon of 261 bp was cloned into the pTV5 vector at Xho I-Xba I site. The second step was to cut out the 3′ end of the gene at the MscI-Xba I site from pFASTBac-GST and clone the 2350 bp fragment into the pTV5 vector from step 1 which already contained the 5′ end of the gene. The resulting vector, FLAG-NDST-2-pTV5, was sequenced using ABI cycle sequencer. The last step was to insert the FLAG-NDST-2-pTV5 construct into the pTVL-ProtA vector. The FLAG-NDST-2-pTV5 vector was cut with Xho I-XbaI and the 2540 bp fragment was ligated into the pTV5L-ProtA vector. Following the CMV promoter, the pTV5L-ProtA vector contains the oncostatin M (OncoM) signal sequence followed by the first two domains of protein A followed by the Xho I and Xba I sites. The oncostatin M gene is published in U.S. Pat. No. 542,812. The resulting vector, A-FLAG-NDST-2-pTV5 was sequenced using ABI cycle sequencer.

Expression of A-FLAG-NDST-2 fusion protein: 20 μg of A-FLAG-NDST-2-pTV5 DNA was electroporated into 1×10⁷ DG44 CHO cells in a 0.4 cm cuvette using a Bio-Rad Gene Pulser (Bio-Rad, Hercules, Calif.). Cells were grown in Protein-Free CHO selection media (Life Technologies) supplemented with Recombulin (500 ug/ml) and L-glutamine (4 mM). For production, 1000 mls of media was seeded with 1×10⁸ cells in a 1 liter spinner. The cells grew for 10 days at 37° C. and the supernatant was harvested.

Purification of A-FLAG-NDST-2 fusion protein: Purification of the CHO expressed A-FLAG-NDST-2 fusion protein was carried out using an anti-FLAG M2 agarose affinity gel from Sigma, St. Louis, Mo. A total of 1 liter of A-FLAG-NDST-2 supernatant was loaded onto a column containing the affinity gel. The column was washed with Phosphate Buffered Saline (PBS), pH 7. The A-FLAG-NDST-2 fusion protein was eluted from the column at pH 3.5 (0.1M glycine and 150 mM NaCl). The eluate was immediately adjusted to pH 7.0 with the addition of 4M Tris, pH 8. Finally, the purified NDST-2 fusion protein was dialyzed against PBS at pH 7.4. The fusion protein had the correct size of approximately 106 kDa by SDS-PAGE analysis and was detected active of N-sulfotransferase activity by using a manual NDST assay [Wei, Z. and Swiedler S. J., (1999) J. Biol. Chem. 274, 1966-1970].

### Example 2

### Preparation of Recombinant Human N-Sulfotransferase Domain of NDST-2 (ST2)

N-sulfotransferase domain of NDST-2 fusion construct: Plasmid containing the coding sequence of the GST-N-sulfotransferase fusion gene (GST-ST2) was generated by subcloning the N-sulfotransferase domain of NDST-2 from the vector pFASTBac-GST-NDST-2. Based upon the human N-sulfotransferase domain of NDST-1 [Sueyoshi, T. et al. (1998) FEBS Letters. 433,211-214], and the alignment of NDST-1 and NDST-2, the DNA fragment consisting of nucleotides 1690-2680 was amplified by PCR utilizing GST-NDST-2 cDNA as a template, the forward primer 5′-ATCAGGATCCCTACAGACCCTTCCTCCTGTCCCA-3′ (SEQ ID NO:5) with a restriction site for BamHI and the reverse primer 5′-GTACGAATTCATCAGCCCAGACTGGAATGCTGCA-3′ (SEQ ID NO:6) with a restriction site for EcoRI. The PCR product was cloned directly into the vector pGEX-4T-3 (Amersham Pharmacia Biotech, Piscataway, N.J.), which contains cDNA of the GST upstream of the restriction site for BamHI. The inserted DNA in pGEX-4T-3 construct was sequenced in both strands by using an ABI cycle sequencer.

Expression of GST-ST2 fusion protein: Mini prepared plasmid GST-ST2 DNA was transformed into E. coli. BL21(DE3)pLysS cells (BL21 cells). The transformed cells were plated onto LB plates containing 50 μg/ml ampicillin and 34 μg/ml Chloramphenicol. The plates were incubated overnight at 37° C., and colonies were isolated. To express GST-ST2 fusion protein, the BL21 cells in LB broth containing 50 μg/ml ampicillin were incubated at 37° C. to a density that OD600 is about 0.5-0.8. Isopropyl-1-thio-β-D-galactose (IPTG) was added into the culture to 0.5 mM final concentration. The culture was transferred to a 30° C. shaker and continuously incubated in 30° C. for 3-4 h. The cells were harvested and cell pellets were collected.

Purification of GST-ST2 fusion protein: Purification of GST-ST2 fusion protein was based on manufacturer's kits and instructions (Amersham Pharmacia Biotech). The cell lysate was applied onto equilibrated Glutathione Sepharose 4B column. The GST-ST2 was eluted by using Glutathione Elution Buffer and then dialyzed against PBS at pH 7.4. The ST2 fusion protein was approximately the expected size of 62 kDa by SDS-PAGE (26 kDa GST and 36 kDa ST2), and active of N-sulfotransferase activity as detected by using a manual NDST assay [Wei, Z. and Swiedler S. J., (1999) J. Biol. Chem. 274, 1966-1970].

### Example 3

### Preparation of Desulfated Heparin-Coated WGA-SPA Beads

Preparation of desulfated heparin-coated WGA-SPA beads was conducted in Reaction Buffer. The Reaction Buffer was also used for SPA assays in Examples 4-6. Components of the Reaction Buffer (pH 7.4) were as follows [Pettersson, I. et al. (1991) J. Biol. Chem. 266, 8044-8049]:

50 mM HEPES

10 mM MnCl₂

10 mM MgCl₂

5 mM CaCl₂

3.5 μM NaF

1% Triton X-100

WGA-SPA beads (50 mg) (RPNQ0001, Amersham Pharmacia Biotech) were incubated with 1 mg of fully desulfated heparin in 10 ml of the Reaction Buffer at 37° C. for 1 h with agitation. The fully desulfated heparin was generated by chemical modification of native pig mucosal heparin (H5284, SIGMA, St. Louis, Mo.) based on the method [Moffat, C. F., et al. (1991) Eur. J. Biochem. 197, 449-459]. The beads were washed 3 times with Reaction Buffer and suspended to a concentration of 40 mg/ml in the same Reaction Buffer containing 0.02% sodium azide. The coated beads were stored at 4° C. until use.

### Example 4

### Solid Phase SPA Assay for N-Sulfotransferase Activity

The assays were conducted at room temperature in Reaction Buffer whose components were described in Example 3. The following components were added into the wells of a 96-well white plate (29444-020, Corning, N.Y.) at a final volume of 100 μl/well.

1. 25 μl of the Reaction Buffer.

2. 25 μl of desulfated heparin-coated beads (20 mg/ml, 500 μg/well).

3. 25 μl of enzyme (ST2, 0.1 mg/ml, 2.5 μg/well or NDST-2, 17.6 μg/ml, 0.44 μg/well) and the plate was incubated on a shaker with slight agitation for 2 min.

4. 25 μl of ³⁵[S]-PAPS (NEG-010, 0.832 mCi/ml, NEN Life Science, Boston, Mass.) (0.4 μCi/ml, 10 nCi/well).

The plate was sealed and incubated for 2-24 h. Radioactivity of the plate was then read in a Packard TopCount microplate scintillation counter (Packard Instrument, Downers Grove, Ill.). The signal-to-noise ratio is the result of counts-per-minute (CPM) in the presence of enzyme divided by counts-per-minute in the absence of enzyme.

### Example 5

### Solution Phase SPA Assay for N-Sulfotransferase Activity

The assays were conducted using conditions similar to the solid phase SPA assay. The following components were added into wells of a 96-well white plate at a final volume of 100 μl/well.

1. 25 μl of desulfated heparin (0.1 mg/ml, 2.5 μg/well).

2. 25 μl of enzyme (ST2, 0.1 mg/ml, 2.5 μg/well or NDST-2, 17.6 μg/ml, 0.44 μg/well).

3. 25 μl of ³⁵[S]-PAPS (0.4 μCi/ml, 10 nCi/well). The plate was mixed by a brief shaking and incubated for 60 min.

4. 25 μl of WGA-SPA beads (20 mg/ml, 500 μg/well).

The plate was sealed and incubated for 2-24 h. Radioactivity of the plate was then read in a Packard TopCount. The signal-to-noise ratio is the result of CPM in the presence of enzyme divided by CPM in the absence of enzyme.

### Example 6

### Method of Screening for Inhibitors of NDST-2

A list of 335 compounds was obtained from a search of the Bristol-Myers Squibb inventoried database using the PHASE program and the bound conformation of PAPS taken from the NDST-1 crystal structure [Kakuta, Y. et al. (1999) J. Biol. Chem. 274, 10673-10676]. The PHASE program is an internally developed program for database searches based on ligand molecular shape and pharmacophroic elements. The test compounds were dissolved in 100% DMSO (D5879, SIGMA) and stored at −20° C. until use. A solid phase SPA assay was used for screening all 335 compounds. The compounds were first added into a 96-well white plate at 10% DMSO final concentration. A serial 2-fold titration was used for measuring IC₅₀ of the interesting compounds. Percentage of inhibition was determined by equation; % Inhibition=(1-cpm with compound/cpm without compound)×100. The inhibitory concentration for 50% of response (IC₅₀) was determined by Microsoft XLfit program (Microsoft, Redmont, Wash.).

### Example 7

### Comparison Between Solid Phase and Solution Phase SPA Assays

The solid phase assay was performed by using fully desulfated heparin that had been immobilized onto the WGA-SPA beads. The pre-coated WGA-SPA beads (500 μg/well), recombinant human sulfotransferase domain of NDST-2 (ST2), as indicated in FIG. 2, and ³⁵[S]-PAPS (10 nCi/well) were added into a 96-well white plate. The plate was incubated at room temperature for 5 h. The solution phase reaction was performed in the same white plate with ST2, free fully-desulfated heparin (2.5 μg/well), and ³⁵[S]-PAPS in the absence of the WGA-SPA beads for 1 h. The WGA-SPA beads were then added and incubated for an additional 4 h. The same concentrations of the beads and PAPS were used in both the solid and the solution phase assays. The plate was read in the TopCount microplate scintillation counter.

The results shown in FIG. 2 indicate that increasing concentrations of the enzyme ST2 (up to 2.5 μg/well) increase the signals (CPMs) of the sulfotransferase activity in both the solid phase and solution phase SPA assays. In the solid phase assay, the average CPM at the concentration of 2.5 μg/well ST2 is 5588 (signal), while the average CPM in the absence of enzyme is 348 (noise); the signal-to-noise ratio is about 16:1. In the solution phase assay, the average CPM at the concentration of 2.5 μg/well ST2 is 2395, while the average CPM in the absence of enzyme is 240; the signal-to-noise ratio is about 10:1.

### Example 8

### Optimization of the Concentration of ³⁵[S]-PAPS

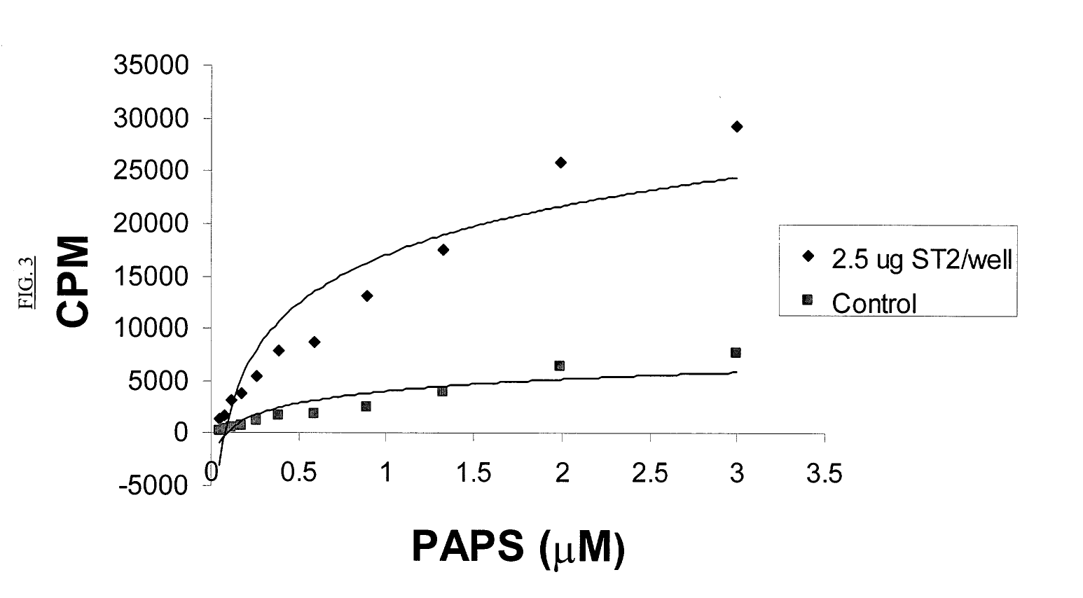

The fully desulfated heparin pre-coated WGA-SPA beads (500 μg/well), recombinant human sulfotransferase domain of NDST-2 (ST2) (2.5 μg/well), and ³⁵[S]-PAPS, as indicated in FIG. 3, were added into a 96-well white plate. The plate was incubated at room temperature for 5 h. The plate was read in the TopCount microplate scintillation counter. The control group was measured in the absence of enzyme ST2. A two-fold titration of PAPS from 3 μM to 3 nM was used to determine the best concentration of PAPS for the highest signal-to-noise ratio.

The results shown in FIG. 3 indicate that increasing the concentration of ³⁵[S]-PAPS from 0 to 1 μM in the presence of 2.5 μg/well ST2 leads to an increase in the signal. The slope of the plot remains constant in this region, suggesting that concentrations of ³⁵[S]-PAPS lower than 1 μM are useful for determining the effects of inhibitors on the enzyme.

### Example 9

### Time-Dependence of ³⁵[S]-PAPS Incorporation

The fully desulfated heparin pre-coated WGA-SPA beads (500 μg/well), recombinant human sulfotransferase domain of NDST-2 (ST2) (2.5 μg/well), and ³⁵[S]-PAPS (10 nCi/well) were added into a 96-well white plate. The plate was incubated at room temperature for the time periods indicated. The plate was read in the TopCount microplate scintillation counter. The control group was measured in the absence of enzyme ST2.

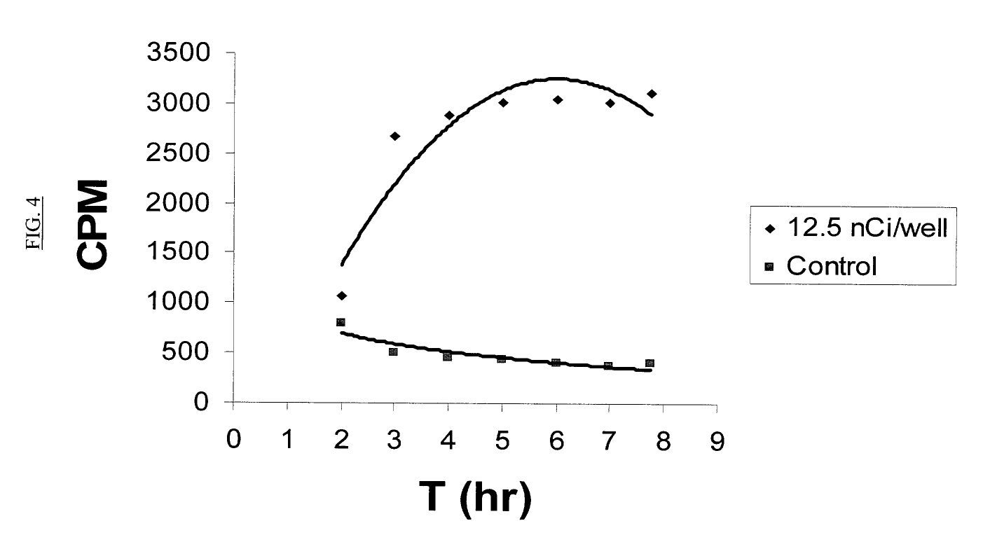

The present kinetic study shown in FIG. 4, indicates that the solid phase SPA assay follows Michaelis-Menten enzyme kinetics in that the enzyme has a rate equation typical of reactions catalyzed by enzymes having a single substrate. The control data indicates that non-specific binding of ³⁵[S] to the bead is low and does not increase during the incubation.

### Example 10

### Determination of Km of PAPS

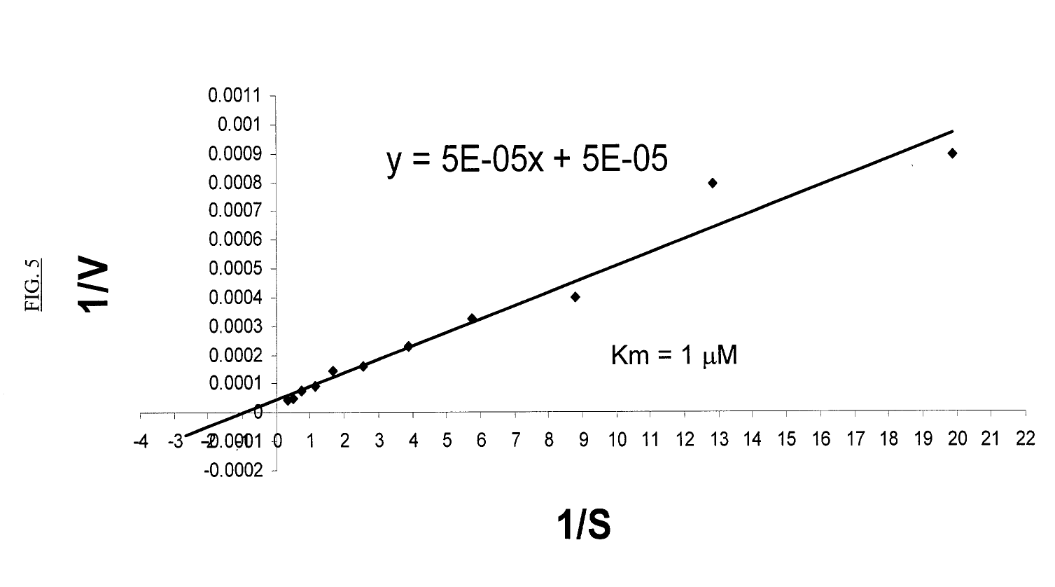

The fully desulfated heparin pre-coated WGA-SPA beads (500 μg/well), recombinant human sulfotransferase domain of NDST-2 (2.5 μg/well), and ³⁵[S]-PAPS, as indicated in FIG. 5, were added into a 96-well white plate. The plate was incubated at room temperature and 5 h. The plate was read in the TopCount scintillation counter. The N-sulfotransferase activity of ST2 is presented as a double-reciprocal plot. The Km of PAPS is determined as a PAPS concentration by using the Michaelis-Menten equation: y=5×10⁻⁵x+5×10⁻⁵.

The results shown in FIG. 5 further demonstrates that the solid phase SPA assay follows Michaelis-Menten enzyme kinetics. The Km value of PAPS based on these results is about 1 μM.

### Example 11

### Identification of Inhibitors by the Solid Phase SPA Assay

Compound A (FIGS. 6A and C) and Compound B (FIGS. 6B and D) in DMSO were added into 96-well white plates at the indicated concentrations. Then, the recombinant human sulfotransferase domain of NDST-2 (ST2, A and B) (2.5 μg/well) or recombinant human wild-type NDST-2 (C and D) (0.4 μg/well) was added to the plates, as well as the fully desulfated heparin pre-coated WGA-SPA beads (500 μg/well) and ³⁵[S]-PAPS (10 nCi/well). The plates were incubated at room temperature for 5 h. The plates were read in the TopCount microplate scintillation counter. Percentage of inhibition was determined by the following equation: % Inhibition=(1-cpm with compound/cpm without compound)×100. IC₅₀ was determined by the Microsoft XLfit program. The IC₅₀ of Compound A is about 0.399 μM (FIG. 6A) and 1.715 μM (FIG. 6C), respectively. The IC₅₀ of Compound B is about 0.520 μM (FIG. 6B) and 2.443 μM (FIG. 6D), respectively. The results suggested that both Compound A and Compound B are N-sulfotransferase inhibitors of NDST-2.

### Example 12

### Testing of Inhibitors by A Manual Sulfotransferase Assay

The manual sulfotransferase assays were conducted basically as described by Pettersson [Pettersson, I. et al. (1991) J. Biol. Chem. 266, 8044-8049; which is hereby incorporated herein by reference]. The following components were added into Eppendorf vials at a final volume of 200 μl/vial.

1. 119 μl of the Reaction Buffer whose components are described in EXAMPLE 3 herein

2. 1 μl of N-acetyl-de-O-sulfated heparin used as a sulfate acceptor (A6039, Sigma, 25 mg/ml, 125 μg/vial).

3. 20 μl of enzyme NDST-2 (17.6 μg/ml, 1.76 μg/vial).

4. 50 μl of test compounds (compound-treated reaction, 25 μM, 6.25 μM/vial) or 50 μl of 10% DMSO in reaction buffer (untreated reaction).

5. 10 μl of ³⁵[S]-PAPS used as a sulfate donor (2 μCi/ml, 100 nCi/vial).

The vials of background cpm do not include enzyme NDST-2. All vials were incubated at 37° C. for 30 min. Reactions were stopped by the addition of 400 μl of 100% ethanol and 35 μl of 3 M sodium acetate into the vials. The samples were placed on dry ice for 5 min and then centrifuged at speed of 14,000 rpm for 5 min. The supernatants were discarded while the pellets were washed twice with 70% ethanol and then dissolved in 100 μl of water. The ³⁵[S]-labeled heparin was separated from unincorporated ³⁵[S]-PAPS by centrifugation through Sephadex G-15 column (Amersham Pharmacia Biotech) at speed of 2,000 rpm for 2 min. The labeled heparin was recovered in the effluents collected in new Eppendorf vials, whereas unincorporated ³⁵[S]-PAPS were retained in the columns. The effluents were transferred into tubes containing 3 ml of EcoLite liquid scintillation cocktail (ICN Biomedicals, Costa Mesa, Calif.). Radioactivity was determined in a Beckman model LS 3800 scintillation counter (Beckman Coulter, Fullerton, Calif.). The percent inhibition of sulfotransferase activity was expressed according to the following formula:

% Inhibition=100%×[1−(cpm in compound-treated reaction−background cpm)÷(cpm in untreated reaction−background cpm)]

The results of these manual assays prove that Compound A and Compound B do, in fact, have N-sulfotransferase inhibitory activity. Moreover, the results of these manual assays further prove that the NDST assay(s) of the present invention is capable of identifying compounds that have N-sulfotransferase inhibitory activity.

The entire disclosure of each document cited (including patents, patent applications, journal articles, abstracts, laboratory manuals, books, or other disclosures) in the Background of the Invention, Detailed Description, and Examples is hereby incorporated herein by reference. Further, the hard copy of the Sequence Listing submitted herewith and the corresponding Computer Readable Form are both incorporated herein by reference in their entireties.

What is claimed is:

1. An assay for measuring the biological activity of an NDST enzyme comprising the step of linking a radiolabeled sulfate with a potentially scintillating particulate bound to a sulfate acceptor in the presence of an NDST enzyme, whereby the radiolabeled sulfate transfers from a sulfate donor to the sulfate acceptor within an activating range of said potentially scintillating particulate to permit emission of a detectable signal.

2. The assay of claim 1 further comprising the step of measuring the signal emitted from the particulates attributable to the N-sulfotransferase activity of the NDST enzyme.

3. The assay of claim 2 wherein the measuring is performed at different time intervals.

4. The assay of claim 2 further comprising the step of determining the amount of N-sulfotransferase activity of the NDST enzyme.

5. The assay of claim 2 wherein said measuring is by scintillation counting.

6. The assay of claim 1 wherein the sulfate donor is ³⁵[S]-adenosine 3′-phosphate 5′-phosphosulfate.

7. The assay of claim 1 wherein the sulfate acceptor is a glycosaminoglycan or a modified version thereof.

8. The assay of claim 1 wherein the sulfate acceptor is a polysaccharide selected from the group consisting of E. coli K5 polysaccharide, N-acetyl desulfated heparin, fully desulfated heparin and heparan sulfate.

9. The assay of claim 1 wherein the potentially scintillating particulates are coated with wheat germ agglutinin.

10. The assay of claim 9 wherein the wheat germ agglutinin binds to the polysaccharide sulfate acceptor.

11. The assay of claim 1 wherein said NDST enzyme comprises a member of the NDST superfamily of enzymes.

12. The assay of claim 11 wherein said NDST is selected from the group consisting of NDST-1, NDST-2, NDST-3, NDST-4, fragments, derivatives, and mutated versions thereof.

13. The assay of claim 1 wherein said assay is a member of the group consisting of a solid phase assay wherein said sulfate acceptor is bound to a potentially scintillating particulate, and a solution phase assay wherein said sulfate acceptor is not bound to a potentially scintillating particulate.

14. A method for screening for compounds which inhibit the biological activity of an NDST enzyme comprising the steps of:
(a) linking a radiolabeled sulfate with a potentially scintillating particulate bound to a sulfate acceptor in the presence of an NDST enzyme, whereby the radiolabeled sulfate transfers from a sulfate donor to said sulfate acceptor within an activating range of said potentially scintillating particulate to permit emission of a detectable signal; and
(b) detecting the light emitted from said particulates in the presence of said test compound to determine N-sulfotransferase inhibition.

15. The method of claim 14 wherein the detecting step includes measuring and comparing the light emitted in the presence of the test compound relative to a control assay run in the absence of said test compound.

16. The method of claim 15 wherein said measuring is by scintillation counting.

17. The method of claim 16 wherein the measuring is performed at different time intervals.

18. The method of claim 15 wherein the sulfate donor is ³⁵[S]-adenosine 3′-phosphate 5′-phosphosulfate.

19. The method of claim 15 wherein the sulfate acceptor is a glycosaminoglycan or a modified version thereof.

20. The method of claim 15 wherein the sulfate acceptor is a polysaccharide selected from the group consisting of E. coli K5 polysaccharide, N-acetyl desulfated heparin, fully desulfated heparin and heparan sulfate.

21. The method of claim 15 wherein the potentially scintillating particulates are coated with wheat germ agglutinin.

22. The method of claim 20 wherein wheat germ agglutinin binds to the polysaccharide sulfate acceptor.

23. The method according to claim 15 wherein said method is a member of the group consisting of a solid phase method wherein said sulfate acceptor is bound to a potentially scintillating particulate, and a solution phase method wherein said sulfate acceptor is not bound to a potentially scintillating particulate.

24. A method of inhibiting biological activity of an NDST enzyme, the method comprising administering to a patient an inhibitory amount of a compound selected from the group consisting of a compound having Formula (I): 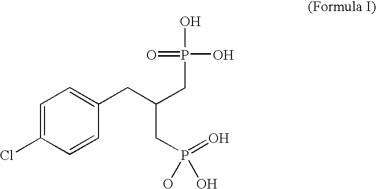
and a compound having Formula (II):

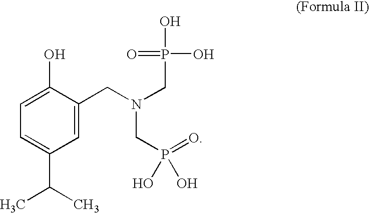

25. A method of inhibiting an allergic response, the method comprising administering to a patient an effective amount of a compound which inhibits the biological activity of an NDST enzyme, whereby said compound is selected from the group consisting of the compound having Formula (I): 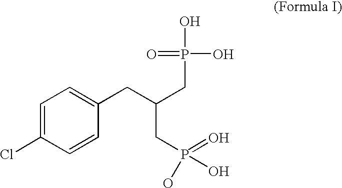
and the compound having Formula (II): [chemical structure]
